(19) United States
(12) Patent Application Publication
(10) Pub. No.: US 2024/0393867 A1
(43) Pub. Date: Nov. 28, 2024

(54) VIRTUAL REALITY SYSTEMS AND METHODS FOR MAGNETIC RESONANCE IMAGING

(71) Applicant: H. LEE MOFFITT CANCER CENTER AND RESEARCH INSTITUTE, INC., Tampa, FL (US)

(72) Inventors: Brian GONZALEZ, Tampa, FL (US); Sarah HOFFE, Bradenton, FL (US); Corrine LEACH, Atlanta, GA (US)

(21) Appl. No.: 18/673,826

(22) Filed: May 24, 2024

Related U.S. Application Data
(60) Provisional application No. 63/504,052, filed on May 24, 2023.

(51) Int. Cl. G06F 3/01; A61B 5/055; A61B 5/08

(52) U.S. Cl. CPC G06F 3/011; A61B 5/055; A61B 5/0803; A61B 5/0816

(57) ABSTRACT

An example system for training a subject to perform an MRI guided procedure includes a virtual reality (VR) headset; a sensor configured to detect respiration; and a controller operably coupled to the VR headset and the sensor, the controller including a processor and a memory, the memory having computer-executable instructions stored thereon that, when executed by the processor, causes the controller to display, on the VR headset, a view from a perspective of a subject lying on a magnetic resonance imaging (MRI) linear accelerator, where the view includes information for assisting the subject in breath-holding to localize a tumor in a desired position.

### CROSS-REFERENCE TO RELATED APPLICATIONS

This application claims the benefit of U.S. provisional patent application No. 63/504,052, filed on May 24, 2023, and titled “NOVEL VIRTUAL REALITY APP FOR TRAINING PATIENTS ON MRI-GUIDED RADIATION THERAPY,” the disclosure of which is expressly incorporated herein by reference in its entirety.

### BACKGROUND

Imaging systems collect data over a period of time to form a representation of a subject. For example, an ordinary digital camera's shutter speed defines the length of time that light is collected by the camera sensor to form the image of the digital camera's subject. Magnetic resonance imaging (MRI) is a common form of imaging that uses magnetic fields and radiofrequency pulses to excite protons in tissue, and thereby produce an image of the inside of a subject's body. A typical MRI system uses a moving gradient field that images sections of the subject sequentially, so that a three-dimensional scan can require a significant amount of time to create. Movement of the subject during this time reduces the accuracy of the resulting MRI image because the data collected during the MRI scan will include data collected from the same tissue located at different points in space during the movement. Accordingly, patient movement can require scans to be repeated. Similarly, movement during MRI guided procedures can require that the procedure be stopped to reposition or restart a procedure. If it is not detected, movement can cause organs to shift and increase unwanted dose to normal tissue.

Improvements to MRI imaging can improve diagnosis and treatment.

### SUMMARY

In some aspects, implementations of the present disclosure include a system including: a virtual reality (VR) headset; a sensor configured to detect respiration; and a controller operably coupled to the VR headset and the sensor, the controller including a processor and a memory, the memory having computer-executable instructions stored thereon that, when executed by the processor, cause the controller to display, on the VR headset, a view from a perspective of a subject lying on a platform of a magnetic resonance imaging (MRI) linear accelerator, wherein the view includes information for assisting the subject in breath-holding to localize a tumor in a desired position.

In some aspects, implementations of the present disclosure include a system, wherein the information includes a dynamic stopwatch to measure a length of breath-holding.

In some aspects, implementations of the present disclosure include a system, wherein the length of breath holding is about 25 seconds.

In some aspects, implementations of the present disclosure include a system, wherein the information includes a first marking that highlights a radiation beam site and a second marking that highlights a target tumor, wherein the first marking and the second marking are overlaid on an MRI image of the subject.

In some aspects, implementations of the present disclosure include a system, wherein the memory has further computer-executable instructions stored thereon that, when executed by the processor, cause the controller to adjust a position of the second marking relative to a position of the first marking in response to a signal from the sensor.

In some aspects, implementations of the present disclosure include a system, wherein the information includes a status of the MRI linear accelerator.

In some aspects, implementations of the present disclosure include a system, wherein the memory has further computer-executable instructions stored thereon that, when executed by the processor, cause the controller to display, on the VR headset, one or more games for assisting the subject in breath-holding.

In some aspects, implementations of the present disclosure include a system, wherein the sensor is a VR controller.

In some aspects, implementations of the present disclosure include a system, wherein the sensor is a respiratory rate monitor.

In some aspects, implementations of the present disclosure include a method including: providing a virtual reality (VR) headset to a subject; providing a sensor configured to detect respiration of the subject; and displaying, on the VR headset, a view from a perspective of the subject lying on a platform of a magnetic resonance imaging (MRI) linear accelerator, wherein the view include information for assisting the subject in breath-holding to localize a tumor in a desired position.

In some aspects, implementations of the present disclosure include a method, wherein the information includes a dynamic stopwatch to measure a length of breath-holding.

In some aspects, implementations of the present disclosure include a method, wherein the length of breath holding is about 25 seconds.

In some aspects, implementations of the present disclosure include a method, wherein the information includes a first marking that highlights a radiation beam site and a second marking that highlights a target tumor, wherein the first marking and the second marking are overlaid on an MRI image of the subject.

In some aspects, implementations of the present disclosure include a method, further including adjusting a position of the second marking relative to a position of the first marking in response to a signal from the sensor.

In some aspects, implementations of the present disclosure include a method, wherein the information includes a status of the MRI linear accelerator.

In some aspects, implementations of the present disclosure include a method, further including displaying, on the VR headset, one or more games for assisting the subject in breath-holding.

In some aspects, implementations of the present disclosure include a method, wherein the one or more games for assisting the subject in breath-holding are displayed prior to the view.

In some aspects, implementations of the present disclosure include a computer-implemented method including: receiving a signal from a sensor configured to detect respiration; and displaying, on a virtual reality (VR) headset, a view from a perspective of a subject lying on a platform of a magnetic resonance imaging (MRI) linear accelerator, wherein the view include information for assisting the subject in breath-holding to localize a tumor in a desired position.

In some aspects, implementations of the present disclosure include a computer-implemented method, further including displaying, on the VR headset, one or more games for assisting the subject in breath-holding.

In some aspects, implementations of the present disclosure include a computer-implemented method, wherein the information includes a first marking that highlights a radiation beam site and a second marking that highlights a target tumor, wherein the first marking and the second marking are overlaid on an MRI image of the subject, the computer-implemented method further including adjusting a position of the second marking relative to a position of the first marking in response to a signal from the sensor.

In some aspects, implementations of the present disclosure include a system including: a virtual reality (VR) headset; a sensor configured to detect respiration; and a controller operably coupled to the VR headset and the sensor, the controller including a processor and a memory, the memory having computer-executable instructions stored thereon that, when executed by the processor, cause the controller to display, on the VR headset, a view from a perspective of a subject lying on a platform of an imaging system, wherein the view include information for assisting the subject in breath-holding to localize a tumor in a desired position.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates an example system block diagram of a system for simulating imaging procedures, according to implementations of the present disclosure.

FIG. 1B illustrates an example system for simulating imaging procedures including a deep-breath belt, according to implementations of the present disclosure.

FIG. 2 illustrates an example method for assisting a subject in performing breath-holding, according to implementations of the present disclosure.

FIG. 3A illustrates an example view including a treatment view and status of an magnetic resonance imaging linear accelerator (MRI LINAC), according to implementations of the present disclosure.

FIG. 3B illustrates an example view including a treatment view and dynamic stopwatch, according to implementations of the present disclosure.

FIG. 4A illustrates an example scene of a game that can be used for training breathing, according to implementations of the present disclosure.

FIG. 4B illustrates an example scene of a game that can be used for training breathing, according to implementations of the present disclosure.

FIG. 5A illustrates an example third-person virtual view of an MRI LINAC, according to implementations of the present disclosure.

FIG. 5B illustrates an example cutaway view of an advanced radiation treatment machine.

FIG. 6 illustrates an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for MRI, and MRI guided radiation therapy, it will become evident to those skilled in the art that the implementations are not limited thereto, but are applicable for any kind of imaging or imaging-guided therapy.

Medical imaging and treatment can require that the subjects of those treatments hold still for extended periods of time, and complete predefined “breath holds” for various periods of time. For example, an advanced radiation treatment is a magnetic resonance imaging (MRI) linear accelerator (LINEAC), which aims a beam of radiation at a patient based on an MRI image (e.g., to destroy a tumor). Tumors are often located in the patients' chest, and the patient's breathing causes the chest (and therefore the tumor to move), preventing the MRI LINAC from successfully targeting the tumor. When patients are unable to hold still or perform the breath holds correctly, the imaging or treatment procedure can be delayed or end prematurely, preventing the patient from receiving effective imaging/treatment, and reducing the number of patients that can be successfully treated by an imaging/treatment system. In addition, patient movement can cause the position of normal tissues to shift, causing unwanted dose in those regions.

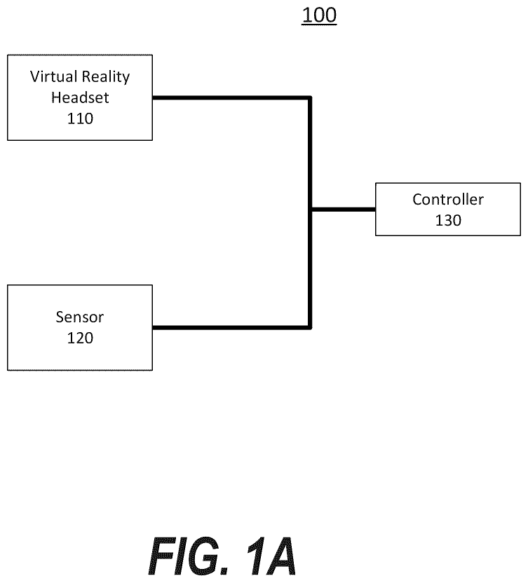

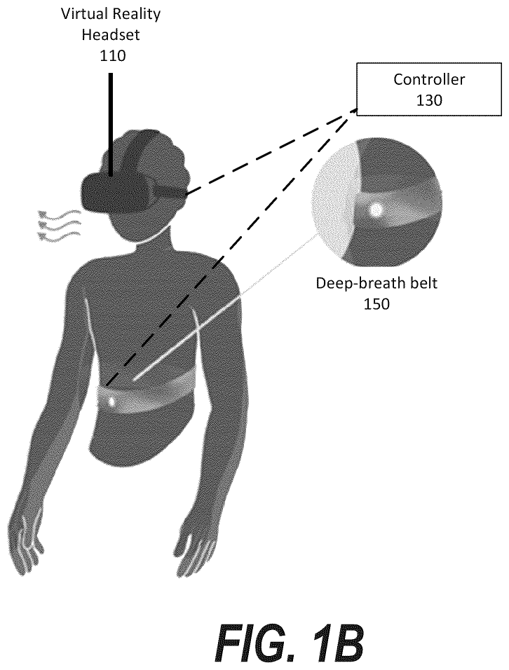

With reference to FIG. 1A, implementations of the present disclosure include systems for developing skills and performing training to improve imaging and treatment procedures. (e.g., advanced radiation treatment using MRI imaging). The implementations of the present disclosure described herein can improve patient training using virtual reality to present views of imaging/treatment procedures, and use sensors to monitor breathing, respiration rate, and/or body position. The sensor data can be output to the patient to provide feedback on the patient's performance performing breath holds, maintaining the correct body position, and/or any other action that the patient may be required to perform during a treatment procedure. This feedback can train a patient to perform the correct breathing patterns during imaging/treatment, and/or acclimatize patients to treatment environments. Because many imaging and treatment centers have limited capacity, improving the performance of each patient improves the number of patients that can be imaged or treated, and thereby improves imaging and treatment of patients using imaging and treatment procedures. feedback, including for example, breathing sensors.

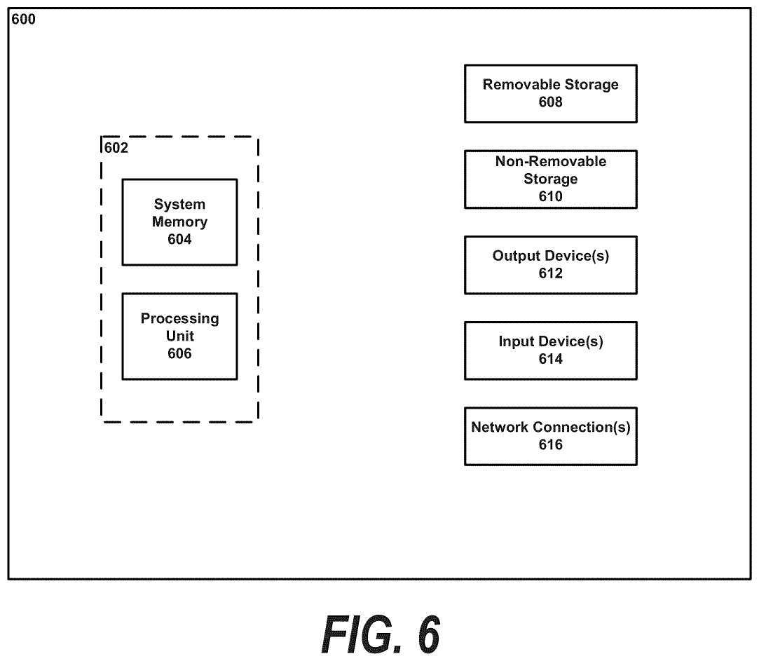

FIG. 1A illustrates an example system 100. The system 100 includes a virtual reality headset 110. As used herein, a “headset” or “virtual reality headset” refers to a display that is configured to present a user with a three-dimensional (stereoscopic) image of a scene, for example by presenting different views to each eye of a user. Virtual reality headsets can optionally be configured so that they block out ambient light, and/or restrict a user's view to what is on one or more screens positioned inside the virtual reality headset. Virtual reality headsets can include audio input and/or outputs to receive and playback audio. Additionally, virtual reality headsets can include one or more computing devices (e.g., the computing device 600 shown in FIG. 6). The present disclosure contemplates that any kind of virtual reality headset can be used, and that different virtual reality headsets used in implementations of the present disclosure can include different combinations of these features.

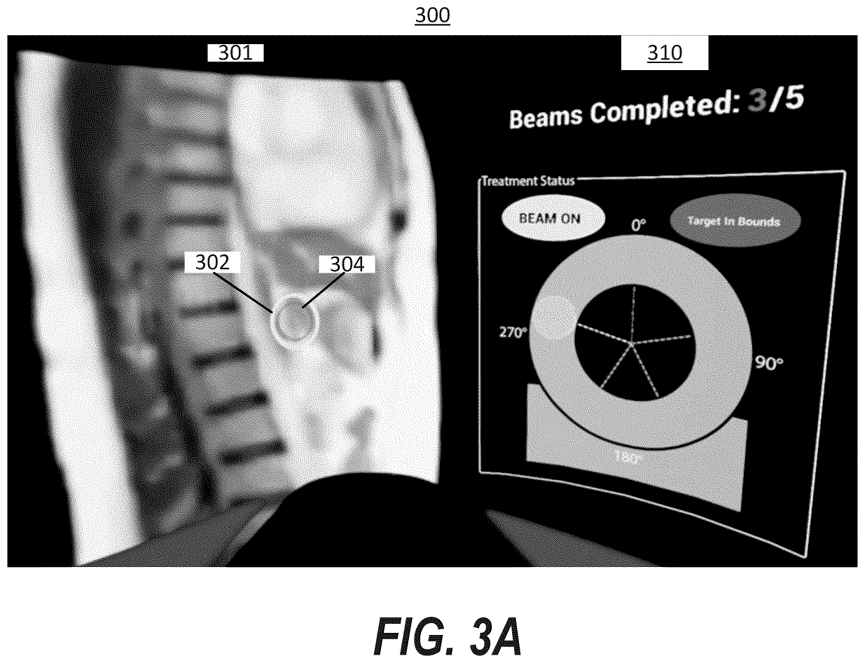

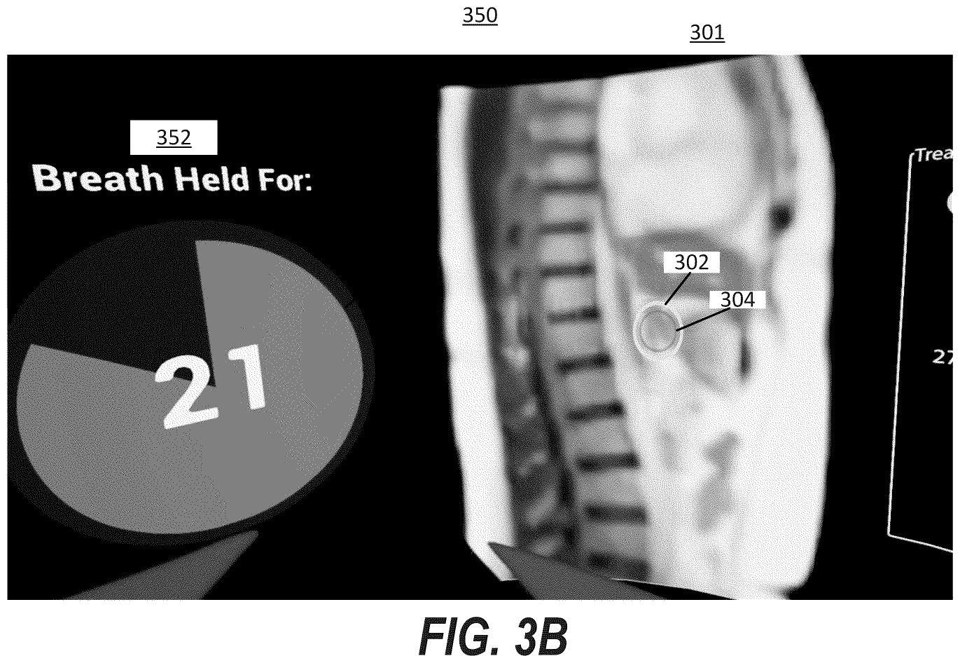

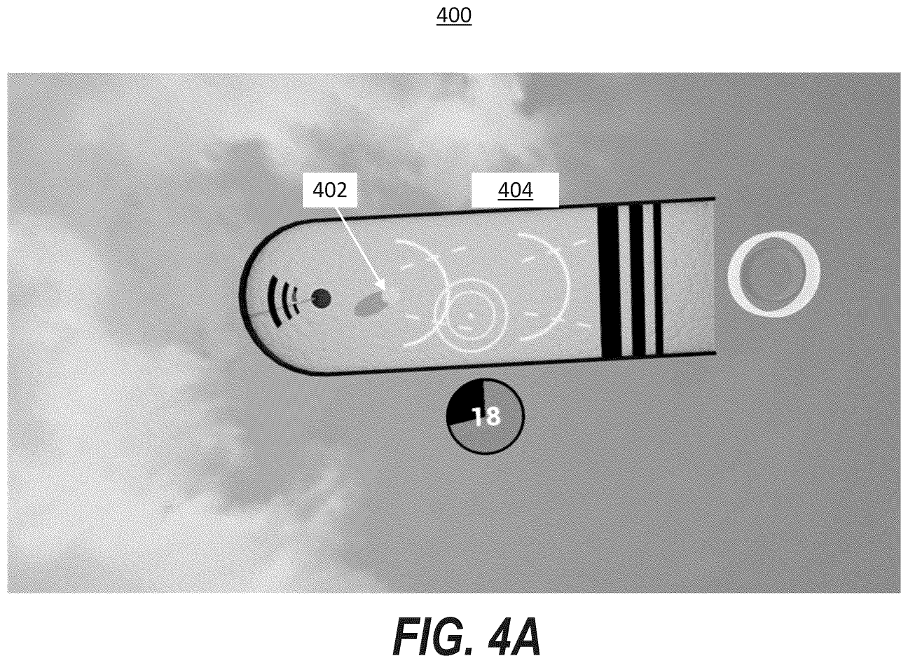

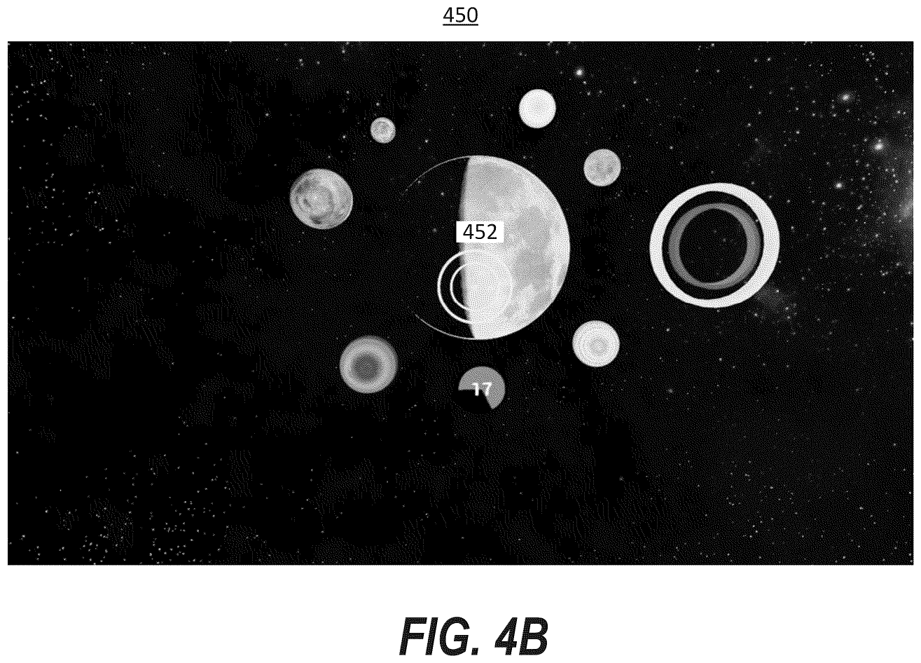

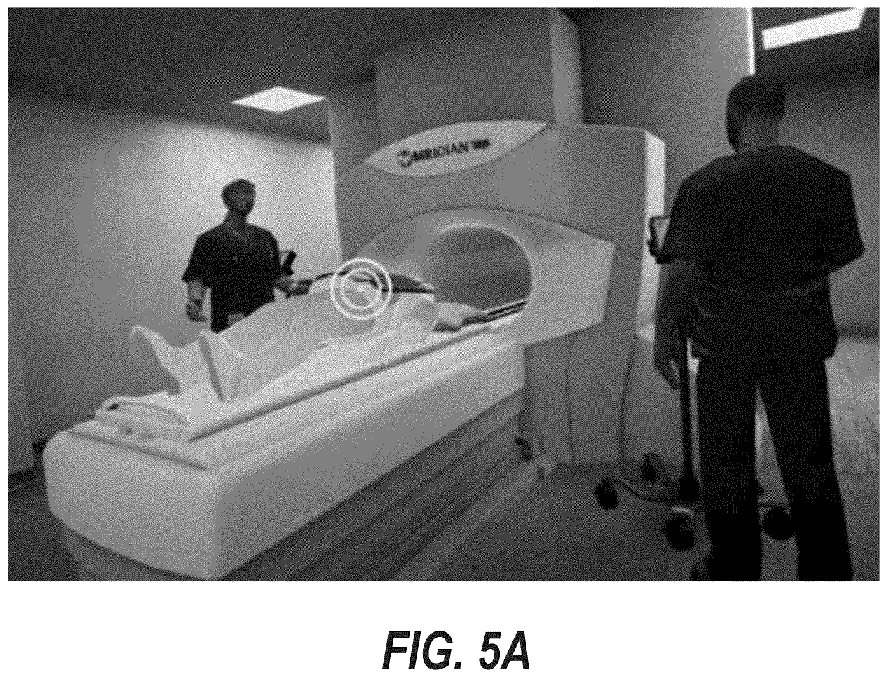

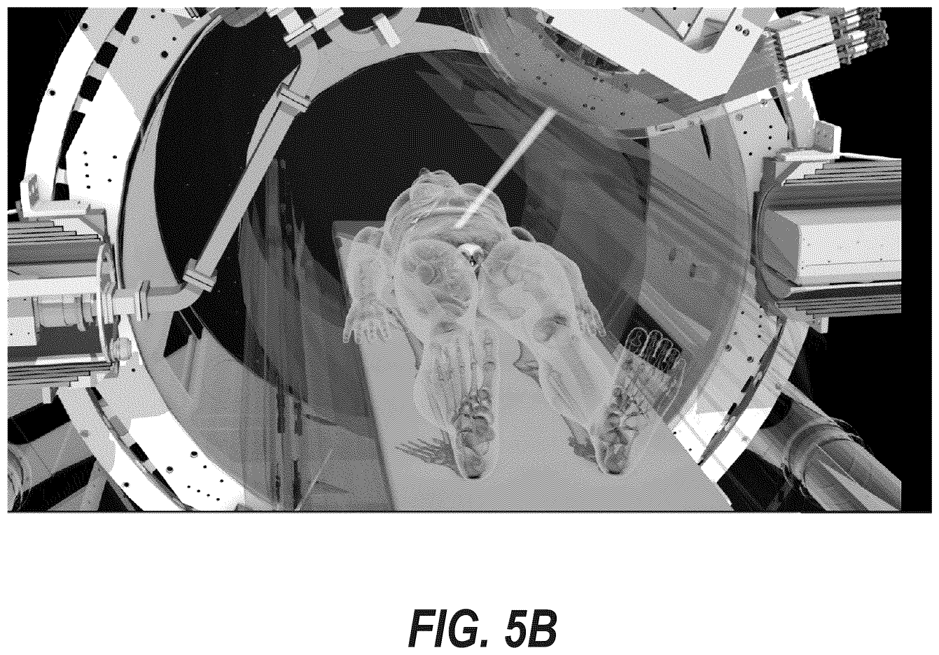

The virtual reality headset 110 can be configured to display virtual environments, interactive games, and information to a user of the virtual reality headset 110. For example, the virtual reality headset 110 can display the scenes 400, 450 of the games illustrated in FIGS. 4A and 4B (as described with respect to examples 1 and 2, below). As additional examples, the virtual reality headset 110 can display virtual reality views like the first example view 300 and second example view 350 shown in FIGS. 3A and 3B. As yet still additional examples, the virtual reality headset 110 can display virtual scenes of a facility for performing advanced radiation treatments, as shown in FIGS. 5A and 5B.

The system further includes a sensor 120. The sensor 120 can optionally be any sensor configured to detect or measure respiration and/or respiratory rate (e.g., a respiratory rate monitor). In some implementations, the sensor 120 is a VR controller. In some implementations, the sensor 120 is a respiratory rate monitor. Optionally, the respiratory rate monitor is a deep breath belt. It should be understood that VR controllers and respiratory rate monitors are provided only as examples. This disclosure contemplates using any sensor configured to detect or measure respiration and/or respiratory rate.

In some implementations, the sensor 120 can be a VR controller. As used herein, a VR controller can be a controller that uses one or more sensors (e.g., infrared, gyroscopic, accelerometer, inertial measurement, etc.) to localize the position of the VR controller in space. For example, a VR controller positioned near a patient's lungs (e.g. placed on chest) can thereby detect/measure respiratory rate by as the controller is moved by the the movement of the lungs/chest/diaphragm. In some implementations, the sensor 120 is a respiratory rate monitor. Optionally, the respiratory rate monitory is a deep breath belt. It should be understood that VR controllers and respiratory rate monitors are provided only as examples. This disclosure contemplates using any sensor configured to detect or measure respiration and/or respiratory rate.

The system further includes a controller 130. The controller can include any or all of the components of the computing device 600 described with reference to FIG. 6. The controller 130 can be coupled to the sensor 120 and/or virtual reality headset 110 using and combination of wired and/or wireless links (e.g., using a network). Optionally, the controller 130 can be implemented as part of the virtual reality headset (e.g., the controller 130 can be a computing device of the virtual reality headset).

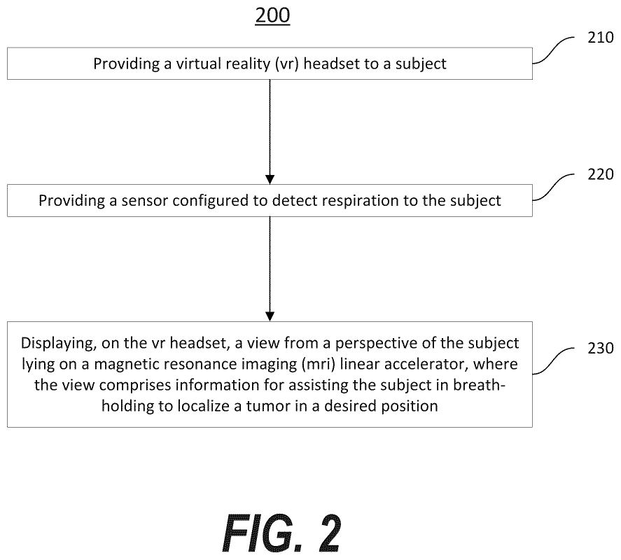

The controller 130 can be configured to perform any of the methods described herein (e.g., the method 200 described with reference to FIG. 2).

For example, in some implementations, the virtual reality headset 110 can be configured to output a dynamic stopwatch to measure a length of breath-holding. An example dynamic stopwatch 352 is shown in FIG. 3B. Optionally, the length of breath holding can be about 25 seconds. It should be understood that the length of 25 seconds is provided only as an example. This disclosure contemplates that the length can be any period of time during which the subject's chest should be held still for imaging and/or treatment purposes.

Alternatively or additionally, the virtual reality headset 110 can be configured to display information including a first marking that highlights a radiation beam site and a second marking that highlights a target tumor. An example first marking 302 and second marking 304 are shown in FIGS. 3A and 3B, where the first marking 302 and the second marking 304 are overlaid on an MRI image of the subject 301. When the subject is properly breath holding, the first marking 302 and the second marking 304 overlap with one another. Thus, the first marking 302 and the second marking 304 provide a visual indication of proper breath holding.

In some implementations, the view can be updated based on movements detected by the sensor 120. For example, the sensor can detect that the target tumor has moved (e.g. due to breathing), and the location of the second marking 304 on the MRI image of the subject can be adjusted relative to the position of the first marking 302 and/or relative to the position of the MRI image of the subject 301.

In some implementations, the virtual reality headset 110 and/or controller 130 can optionally further be configured to display one or more games on the virtual reality headset 110 for assisting the subject in breath-holding. Example games are illustrated in FIGS. 4A and 4B. In FIG. 4B, the subject's breath holding controls the distance a golf ball travels on a putting green. In FIG. 4A, the subject's breath holding causes the moon to appear in a night sky scene.

With reference to FIG. 1B, an example system is shown according to implementations of the present disclosure. The system includes a deep breath belt 150 that can be operably coupled to the virtual reality headset 110 and/or controller 130. The deep breath belt includes one or more sensors to measure respiratory rate based on the motion of the patient inside the deep breath belt 150.

With reference to FIG. 2, implementations of the present disclosure include methods of displaying a virtual reality image based on a subject's breathing. The methods described herein can be used to train subjects to control their breathing and/or any other body movements to avoid movement during a medical imaging process.

At step 210, the method includes providing a virtual reality (VR) headset to a subject. Example virtual reality headsets that can be used in implementations of the present disclosure are shown and described with reference to FIGS. 1A and 1B. It should be understood that any type of virtual reality headset can be used in the methods described herein.

At step 220 the method includes providing a sensor configured to detect respiration of the subject. The sensor can optionally include the sensor 120 described with reference to FIG. 1A and/or a deep breath belt 150 as shown in FIG. 1B.

At step 230 the method includes displaying, on the VR headset, a view from a perspective of a subject inside an imaging system. Optionally, the imaging system is a magnetic resonance imaging (MRI) system. It should be understood that MRI systems are provided only as an example. This disclosure contemplates using the systems and methods described herein with other imaging modalities, particularly where breath holding is desirable during imaging. Optionally, the imaging system is part of a treatment system, e.g. an imaging-guided treatment system. One such example is an MRI linear accelerator (LINAC) for image-guided radiation therapy. For example, the view can be a view from a perspective of the subject lying on a platform of an MRI LINAC. The view can include information for assisting the subject in breath-holding to localize a tumor in a desired position. Example views 300, 350 that can be displayed at step 230 are shown and described with reference to FIGS. 3A and 3B.

Optionally, the information can include a dynamic stopwatch (e.g., the dynamic stopwatch 352 shown in FIG. 3B) to measure a length of breath-holding (e.g., about 25 seconds).

Alternatively or additionally, the information can include a first marking that highlights a radiation beam site and a second marking that highlights a target tumor, where the first marking and the second marking are overlaid on an MRI image of the subject (e.g., as illustrated by the example first marking 302 and second marking 304 shown in FIGS. 3A and 3B). In some implementations, the method can include adjusting a position of the second marking relative to a position of the first marking in response to a signal from the sensor.

The present disclosure contemplates that any information from the method described herein can be displayed. For example, in some implementations, the information includes a status of the MRI linear accelerator (e.g., as illustrated by the example status 310 shown in FIG. 3A). Alternatively or additionally, the method can include displaying, on the VR headset, one or more games for assisting the subject in breath-holding (e.g., the games illustrated in FIGS. 4A and 4B). The present disclosure contemplates that the games can be displayed in different orders during the method. For example, the games can be displayed prior to the view. In other words, the games can be presented to the subject in order to familiarize the subject with the breath holding exercise prior displaying the view from the subject's perspective within the imaging system. Alternatively or additionally, the games can be displayed after the view, or at intervals interspersed with the view.

It should be understood that in some implementations, the methods described with reference to FIG. 2 can be implemented as computer-implemented methods, for example by using one or more computing devices as described herein.

FIGS. 3A and 3B illustrate example views that can be generated and/or output using the systems and methods described herein. For example the views shown in FIGS. 3A and 3B can be output using controller 130 and/or virtual reality headset 110 of FIGS. 1A-1B, or as part of the method 200 shown in FIG. 2.

FIG. 3A illustrates a first example view 300 according to implementations of the present disclosure. The first example view 300 includes an MRI image of the subject 301 including first marking 302 that highlights a radiation beam site and a second marking 304 that highlights a target tumor. The first example view 300 further includes a status 310 of the MRI linear accelerator.

FIG. 3B illustrates a second example view 350. The second example view 350 includes the treatment view, first marker, and second marker shown and described in FIG. 3A. The second example view 350 further illustrates a dynamic stopwatch 352 that can measure and/or display a length of breath-holding.

It should be appreciated that the logical operations described herein with respect to the various figures may be implemented (1) as a sequence of computer implemented acts or program modules (i.e., software) running on a computing device (e.g., the computing device described in FIG. 6), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 6, an example computing device 600 upon which the methods described herein may be implemented is illustrated. It should be understood that the example computing device 600 is only one example of a suitable computing environment upon which the methods described herein may be implemented. Optionally, the computing device 600 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 600 typically includes at least one processing unit 606 and system memory 604. Depending on the exact configuration and type of computing device, system memory 604 may be volatile (such as random access memory (RAM)), non-volatile (such as read-only memory (ROM), flash memory, etc.), or some combination of the two. This most basic configuration is illustrated in FIG. 6 by box 602. The processing unit 606 may be a standard programmable processor that performs arithmetic and logic operations necessary for operation of the computing device 600. The computing device 600 may also include a bus or other communication mechanism for communicating information among various components of the computing device 600.

Computing device 600 may have additional features/functionality. For example, computing device 600 may include additional storage such as removable storage 608 and non-removable storage 610 including, but not limited to, magnetic or optical disks or tapes. Computing device 600 may also contain network connection(s) 616 that allow the device to communicate with other devices. Computing device 600 may also have input device(s) 614 such as a keyboard, mouse, touch screen, etc. Output device(s) 612 such as a display, speakers, printer, etc. may also be included. The additional devices may be connected to the bus in order to facilitate communication of data among the components of the computing device 600. All these devices are well known in the art and need not be discussed at length here.

The processing unit 606 may be configured to execute program code encoded in tangible, computer-readable media. Tangible, computer-readable media refers to any media that is capable of providing data that causes the computing device 600 (i.e., a machine) to operate in a particular fashion. Various computer-readable media may be utilized to provide instructions to the processing unit 606 for execution. Example tangible, computer-readable media may include, but is not limited to, volatile media, non-volatile media, removable media and non-removable media implemented in any method or technology for storage of information such as computer readable instructions, data structures, program modules or other data. System memory 604, removable storage 608, and non-removable storage 610 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 606 may execute program code stored in the system memory 604. For example, the bus may carry data to the system memory 604, from which the processing unit 606 receives and executes instructions. The data received by the system memory 604 may optionally be stored on the removable storage 608 or the non-removable storage 610 before or after execution by the processing unit 606.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

A study was performed of an example implementation of the present disclosure on patients receiving respiratory gated Magnetic Resonance Imaging guided Radiation Therapy (MRIgRT) for abdominal targets must hold their breath for >25 seconds at a time. The example implementation successfully educated, trained, and reduced anxiety and discomfort in patients preparing to receive MRIgRT.

The example implementation included mini-games to help orient patients to using the VR device and to train patients on breath-holding. Users can be introduced to the MRI LINAC vault and practice breath-holding during MRIgRT. In this quality improvement project, patients with pancreatic cancer who were eligible for MRIgRT and clinic personnel tested the VR app for feasibility, acceptability, and potential efficacy for training patients on using breath-holding during MRIgRT.

The new VR app experience was tested by 19 patients and 67 clinic personnel. The experience was completed on average in 18.6 minutes (SD=5.4) by patients and in 14.9 (SD=3.5) minutes by clinic personnel. Patients reported the app was “extremely helpful” (58%) or “very helpful” (32%) for learning breath-holding used in MRIgRT and “extremely helpful” (28%) or “very helpful (50%) for reducing anxiety. Patients and clinic personnel also provided qualitative feedback on improving future versions of the VR app.

The VR app was feasible and acceptable for training patients on breath-holding for MRIgRT. Patients eligible for MRIgRT for pancreatic cancer.

Magnetic Resonance Imaging guided Radiation Therapy (MRIgRT) is a therapy that integrates adaptive therapy and breath-holding to deliver higher biologically effective doses of radiation to tumors and minimize toxicity to healthy tissue. [1] However, MRI-related discomfort and distress are major barriers to MRIgRT. Up to 15% of patients experience MRI-related claustrophobia,2 14% cannot complete MRI due to severe MRI-related anxiety, [3] and 29-65% report MR-related complaints. [4,5]

Patients receiving abdominal MRIgRT cannot be easily sedated because they must intermittently hold a deep breath so that gross tumor volume can be targeted. During MRIgRT, patients are shown an image with one circle depicting the static tumor target and another circle depicting their respiratory motion. Patients are asked to take and hold a deep breath so that the circles are superimposed for as long as they feel comfortable. Practice with this procedure can reduce the time to completion, with one study finding 13% faster final MRIgRT treatments than first treatments.6 Thus, training for breath-holding may reduce the time needed to complete MRIgRT, increase willingness to undergo MRIgRT, and reduce MRIgRT-related distress.

Mock MRI training [7] and mobile apps[8] have shown promise for preparing children for MRI; however, these approaches may have limited impact for two reasons. Mock MRI is typically resource-intensive. Smartphone/tablet apps are portable and require fewer resources but are also less immersive. Virtual reality (VR), defined as “computer-generated, interactive, and highly vivid environments” that are multi-sensory and immersive,9 overcomes both limitations. One study compared receiving simulated MRI in VR vs. mock MRI. Patients felt that mock simulation was more realistic than VR, but patients in both groups reported similar levels of discomfort and anxiety, [10] suggesting VR achieves near-realistic experiences of MRI. [11]

The example implementation included immersive and gamified experiences to educate, train, and reduce anxiety in patients preparing to receive MRIgRT. The example implementation was acceptable to patients and clinic personnel, feasible for use in preparing patients for MRIgRT, and efficacious for training patients on deep inspiration breath-holding.

This quality improvement project was deemed exempt from IRB review. A multi-disciplinary project team included two pancreatic cancer survivors and experts in radiation oncology, VR development, video game graphic design, behavioral medicine, and digital health technology. A design team received feedback from the larger team and met weekly to review progress on the app's features, gamification, user experience, and user interface.

Designers developed the virtual environment and 3-dimensional assets to be included in the environment. Audio files were developed to play ambient sounds, background music, instructions, and responses to user actions. The environment and assets were integrated using the Unreal Engine 4.26. The app was deployed on Meta Quest 2 VR headsets, tested, and iteratively refined to ensure the app was immersive, realistic, and easy to use. Lastly, the app was deployed for testing with patients and clinic personnel.

The study invited consecutive patients with pancreatic cancer eligible for MRIgRT and clinic personnel to use the VR app during a trial period. Prior to simulation, users sat upright on a reclining chair and were familiarized with the app and controls. Users then lied down horizontally before playing two mini-games shown in FIGS. 4A and 4B to promote relaxation and training on the breath-holding technique. Respiratory movement was tracked via a VR controller placed on the user's chest (e.g., the deep breath belt 150 shown in FIG. 1B) to show how to superimpose the breathing circle over the static target circle using breath-holding. The mini-games asked users to hold their breath for 25 seconds to control objects in the game. An example game includes the scene 400 shown in FIG. 4A, where the 25 second breath hold controls the distance a golf ball 402 travels on a putting green 404. Another example game includes the scene 450 shown in FIG. 4B, where the 25 second breath hold causes the moon 452 to appear in a night sky scene. The mini games and scenes 400, 450 described herein are intended only as non-limiting examples, and other breath-training mini games can be used in various implementations of the present disclosure.

Next, users were introduced to the staff and equipment in an MRI vault. Users were then shown a view from the perspective of a patient lying on an MRI LINAC. FIGS. 3A and 3B show example views 300, 350 from the user's position while receiving MRIgRT, including breath-holding to localize the tumor in its proper deep breath hold position.

Before and after using the VR app, patients were asked to report how anxious they would feel about going through MRIgRT. After using the app, patients were also asked whether the app was helpful for understanding breath-holding and whether the app was helpful for decreasing their anxiety. After using the VR app, clinic personnel were asked whether the app was helpful for training patients on breath holding.

### Quantitative Results

The study included measures of quantitative results. Of the 22 patients invited to use the VR app, 19 (86%) agreed, including 9 (47%) females and 10 (53%) males. Patients who reported their ages were <50 (n=3, 17%), 50-70 (n=6, 33%), and 71-80 years old (n=9, 47%). Of the 68 clinic personnel invited to use the VR app, 67 (99%) agreed. Clinic personnel who reported their ages were <30 (n=12, 19%), 30-40 (n=26, 41%), 41-50 (n=13, 20%), and >51 years old (n=13, 20%). Clinical roles were nurse (n=3, 4%), physicist (n=6, 9%), radiation oncologist (n=3, 4%), radiation therapist (n=9, 13%), dosimetrist (n=3, 4%), advanced practice professional (n=3, 4%), fellow/resident (n=13, 19%), clinical trial coordinator (n=5, 7%), and other (n=22, 33%).

On average, patients completed the app experience in 18.6 minutes (SD=5.4), and clinic personnel completed the app experience in 14.9 minutes (SD=3.5). Before starting the VR app, 42% patients reported they felt at least “somewhat” anxious about MRIgRT. After completing the app, MRIgRT-related anxiety had not changed, with 42% reporting they were at least “somewhat anxious” about MRIgRT. Patients reported the VR app was “extremely helpful” (58%) or “very helpful” (32%) for learning breath-holding for MRIgRT. Of the 18 patients who replied to the question asking how much the VR app helped with decreasing anxiety, 28% reported it was “extremely helpful,” 50% reported it was “very helpful,” and 22% reported it was “somewhat helpful.” Of the 66 clinic personnel who replied to the question asking whether the app would be helpful for training patients on breath-holding for MRIgRT, 68% reported it was “extremely helpful,” and 29% reported it was “very helpful.”

In brief interviews, commonly reported themes were identified related to usability and potential impact. First, many users reported that controllers for tracking users' breathing sometimes did not detect when the user was holding their breath. Second, users reported that in-app verbal instructions were sometimes unclear or given too quickly, reducing their understanding of the instructions. Third, some users had difficulty finding instructions/interactions that were not centered in the view and others reported difficulty rotating their heads to look at various objects due to comorbidities. Clinic personnel reported the app would likely increase patient understanding of the experience of MRIgRT. Users reported the app would help increase patient self-efficacy in their ability to receive MRIgRT and participate in breath-holding. One patient reported, “I really didn't know what to expect, but this really helps you because it tells me what to do, what to expect.”

### DISCUSSION

Immersive VR technologies can improve the patient experience, increase patient satisfaction, decrease distress, and prepare patients for new procedures. The VR app, designed with input from patients and a multidisciplinary team, used two mini-games to orient patients to breath-holding before introducing patients to the MRI linac. Users also practiced breath-holding while in the VR MRI linac bore. Most patients felt the prototype decreased their anxiety and taught them to use breath-holding for >25 seconds. Similarly, clinic personnel felt the experience would increase the preparation and readiness for first-time MRI linac users.

Patients generally reported the app was helpful in reducing anxiety, but MRIgRT-related anxiety did not change from before to after using the VR app. This may reflect a lack of specificity in how the questions were asked. In reporting MRIgRT-related anxiety, patients may have focused on anxiety related to whether MRIgRT would be efficacious rather than on their self-efficacy for completing MRIgRT. Future studies should use validated questionnaires to assess change in specific constructs, such as self-efficacy for completing breath-holding and receiving MRIgRT.

User feedback helped identify VR app improvements. Users reported that the controller was not sufficiently sensitive for breath-tracking. Also, the app's instructions were too rapid or unclear. Lastly, users also reported difficulty moving their head during some mini-games. Future versions of the VR app must identify alternate strategies for monitoring respiration, provide slower instructions with visual cues to enhance understanding,¹³ personalize the pace of instructions and feedback to users, and accommodate the needs of patients with limited mobility. The average length of the VR experience was 18.6 minutes, but a shorter experience time may better accommodate busy clinics.

In conclusion, the example implementation of the present disclosure demonstrated feasibility and acceptability in patients and clinic personnel. Users reported the app was easy to use and helpful for learning breath-holding and preparation for MRIgRT.

### Example 2

A study was performed of an example implementation of the present disclosure for preparing subjects for advanced radiation treatments. Advanced radiation treatments can improve patient outcomes, but can require patients to develop complicated skills for the advanced radiation treatments to be effectively used on the patients. An example of an advanced radiation treatment is an MRI linear accelerator, or “MRI linac.”

An MRI linac requires the patients to have the skills to perform certain actions while the MRI linac procedure is performed. For example, an MRI linac procedure can require that the patient lay still within the machine for 60-90 minutes, and perform intermittent breath holds for 25 seconds or more. Alternatively or additionally, the MRI linac can form an enclosed or partially-enclosed tube the patient is positioned inside. Patients commonly lack the skills to physically complete MRI linac procedures and other advanced radiation treatments.

Implementations of the present disclosure include systems and methods that can be used by patients to develop the skills required to successfully act as a subject in an advanced radiation treatment system. An example implementation of the present disclosure includes a VR headset, where the training can begin when the patient puts on the headset. Training includes “mini games” that can be used to train the patients breathing for the breath hold component of an advanced radiation treatment procedure. Example mini games that can be used include the games shown and described with reference to FIGS. 4A and 4B herein. Mini games can also be used to help the patient relax at different points in the training process (e.g., at the beginning).

Optionally, the training can include a patient being positioned in a treatment room and/or in a real or simulated advanced radiation treatment machine. For example, the patient can be placed in the treatment room after performing the mini games. In some implementations, the system can be configured to provide a virtual reality view of the treatment or treatment environment to the patient. For example, the virtual view can include the first example view 300 and/or second example view 350 shown in FIGS. 3A and 3B. The virtual view can be configured to simulate the patient entering the advanced radiation treatment machine (e.g., an MRI machine). Alternatively or additionally, the patient can be positioned in the MRI machine while viewing a virtual reality view.

Optionally, the training can further include third-person views of patients being positioned in advanced radiation treatment machines. Example third-person views of patients in advanced radiation treatment machines are shown in FIG. 5A, which illustrates an example virtual view of an MRI Linac, and FIG. 5B which illustrates an example cutaway view of an advanced radiation treatment machine.

As used herein, the terms “about” or “approximately” when referring to a measurable value such as an amount, a percentage, and the like, is meant to encompass variations of ±20%, 10%, 5%, or +1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

### REFERENCES

1. Hall W A, Paulson E, Li X A, et al: Magnetic resonance linear accelerator technology and adaptive radiation therapy: An overview for clinicians. CA: a cancer journal for clinicians 72:34-56, 2022
2. Dewey M, Schink T, Dewey C F: Claustrophobia during magnetic resonance imaging: cohort study in over 55,000 patients. Journal of Magnetic Resonance Imaging: An Official Journal of the International Society for Magnetic Resonance in Medicine 26:1322-1327, 2007
3. Katznelson R, Djaiani G N, Minkovich L, et al: Prevalence of claustrophobia and magnetic resonance imaging after coronary artery bypass graft surgery. Neuropsychiatr Dis Treat 4:487-93, 2008
4. Tetar S, Bruynzeel A, Bakker R, et al: Patient-reported outcome measurements on the tolerance of magnetic resonance imaging-guided radiation therapy. Cureus 10, 2018
5. Kluter S, Katayama S, Spindeldreier C K, et al: First prospective clinical evaluation of feasibility and patient acceptance of magnetic resonance-guided radiotherapy in Germany. Strahlentherapie und Onkologie 196:691-698, 2020
6. Hoffe S, Madey K, Gonzalez B D, et al: Adaptive Stereotactic Body Radiation Therapy (SBRT) on an MRI Linear Accelerator for Patients with Pancreatic Cancer: Does Treatment Time Efficiency Improve by Therapy Completion?, Annual Meeting of the American Radium Society, 2022, pp S29-S29
7. de Bie HMA, Boersma M, Wattjes M P, et al: Preparing children with a mock scanner training protocol results in high quality structural and functional MRI scans. European Journal of Pediatrics 169:1079-1085, 2010
8. Pua EPK, Barton S, Williams K, et al: Individualised MRI training for paediatric neuroimaging: A child-focused approach. Developmental Cognitive Neuroscience 41:100750, 2020
9. Boyd D E, Koles B: An introduction to the special issue “virtual reality in marketing”: definition, theory and practice, Elsevier, 2019, pp 441-444
10. Nakarada-Kordic I, Reay S, Bennett G, et al: Can virtual reality simulation prepare patients for an MRI experience? Radiography 26:205-213, 2020
11. Brown RKJ, Petty S, #039, et al: Virtual Reality Tool Simulates MRI Experience. Tomography 4:95-98, 2018
12. Hudson D M, Heales C, Vine S J: Scoping review: How is virtual reality being used as a tool to support the experience of undergoing Magnetic resonance imaging? Radiography 28:199-207, 2022
13. Nielsen J: Enhancing the explanatory power of usability heuristics, Proceedings of the SIGCHI conference on Human Factors in Computing Systems, 1994, pp 152-158

1. A system comprising:
a virtual reality (VR) headset;
a sensor configured to detect respiration; and
a controller operably coupled to the VR headset and the sensor, the controller comprising a processor and a memory, the memory having computer-executable instructions stored thereon that, when executed by the processor, cause the controller to display, on the VR headset, a view from a perspective of a subject lying on a platform of a magnetic resonance imaging (MRI) linear accelerator, wherein the view comprises information for assisting the subject in breath-holding to localize a tumor in a desired position.

2. The system of claim 1, wherein the information comprises a dynamic stopwatch to measure a length of breath-holding.

3. The system of claim 2, wherein the length of breath holding is about 25 seconds.

4. The system of claim 1, wherein the information comprises a first marking that highlights a radiation beam site and a second marking that highlights a target tumor, wherein the first marking and the second marking are overlaid on an MRI image of the subject.

5. The system of claim 4, wherein the memory has further computer-executable instructions stored thereon that, when executed by the processor, cause the controller to adjust a position of the second marking relative to a position of the first marking in response to a signal from the sensor.

6. The system of claim 1, wherein the information comprises a status of the MRI linear accelerator.

7. The system of claim 1, wherein the memory has further computer-executable instructions stored thereon that, when executed by the processor, cause the controller to display, on the VR headset, one or more games for assisting the subject in breath-holding.

8. The system of claim 1, wherein the sensor is a VR controller.

9. The system of claim 1, wherein the sensor is a respiratory rate monitor.

10. A method comprising:
providing a virtual reality (VR) headset to a subject;
providing a sensor configured to detect respiration of the subject; and
displaying, on the VR headset, a view from a perspective of the subject lying on a platform of a magnetic resonance imaging (MRI) linear accelerator, wherein the view comprises information for assisting the subject in breath-holding to localize a tumor in a desired position.

11. The method of claim 10, wherein the information comprises a dynamic stopwatch to measure a length of breath-holding.

12. The method of claim 11, wherein the length of breath holding is about 25 seconds.

13. The method of claim 10, wherein the information comprises a first marking that highlights a radiation beam site and a second marking that highlights a target tumor, wherein the first marking and the second marking are overlaid on an MRI image of the subject.

14. The method of claim 13, further comprising adjusting a position of the second marking relative to a position of the first marking in response to a signal from the sensor.

15. The method of claim 10, wherein the information comprises a status of the MRI linear accelerator.

16. The method of claim 10, further comprising displaying, on the VR headset, one or more games for assisting the subject in breath-holding.

17. The method of claim 16, wherein the one or more games for assisting the subject in breath-holding are displayed prior to the view.

18. A computer-implemented method comprising:
receiving a signal from a sensor configured to detect respiration; and
displaying, on a virtual reality (VR) headset, a view from a perspective of a subject lying on a platform of a magnetic resonance imaging (MRI) linear accelerator, wherein the view comprises information for assisting the subject in breath-holding to localize a tumor in a desired position.

19. The computer-implemented method of claim 18, further comprising displaying, on the VR headset, one or more games for assisting the subject in breath-holding.

20. The computer-implemented method of claim 18, wherein the information comprises a first marking that highlights a radiation beam site and a second marking that highlights a target tumor, wherein the first marking and the second marking are overlaid on an MRI image of the subject, the computer-implemented method further comprising adjusting a position of the second marking relative to a position of the first marking in response to a signal from the sensor.
